(19) United States
(12) Patent Application Publication
(10) Pub. No.: US 2016/0011174 A1
(43) Pub. Date: Jan. 14, 2016

(54) Assay for Determining the Cell Number in Cultured Cells

(71) Applicant: KEELE UNIVERSITY, Staffordshire (GB)

(72) Inventors: Alicia EL-HAJ, Staffordshire (GB); Thomas W. E. CHIPPENDALE, Staffordshire (GB); David SMITH, Staffordshire (GB); Patrik SPANEL, Staffordshire (GB)

(21) Appl. No.: 14/770,547

(22) PCT Filed: Feb. 28, 2013
(86) PCT No.: PCT/EP2013/054025

§ 371 Date: Aug. 26, 2015

(51) Int. Cl. G01N 33/50

(52) U.S. Cl. CPC G01N 33/5008; G01N 2500/10

(57) ABSTRACT

A method for determining the number of cells in an in vitro culture of cells is disclosed, the method comprising culturing cells in the presence of dimethyl sulphoxide (DMSO) for a period of time sufficient for the cells to produce dimethyl sulphide (DMS), measuring the DMS produced by the cultured cells.

### FIELD OF THE INVENTION

The present invention relates to cell culture methods, methods of determining the number of cells in a cell culture, methods of determining the ability of a test compound or test condition to kill cells or enhance cell proliferation.

### BACKGROUND TO THE INVENTION

### Acetaldehyde Metabolism and Disease

Aldehyde dehydrogenase (ALDH) enzymes are responsible for the metabolism of aldehydes, including acetaldehyde (AA), and are linked to disease. The toxic volatile compound acetaldehyde (AA) is an intermediary of human ethanol metabolism; the proposed mechanism being that ethanol is oxidised to AA, which is then further oxidised to acetate via enzyme-mediated reactions (Ref 1). However, AA may also be formed by alternative mechanisms, including lipid peroxidation (Ref 2). Aldehyde dehydrogenase (ALDH) enzymes are thought to be primarily responsible for the oxidation/detoxification of aldehydes, including AA, in conjunction with the coenzyme nicotinamide adenine dinucleotide (NAD+), to form the corresponding carboxylic acids. There are 19 ALDH isozymes expressed in human cells, which have varying reaction efficiencies depending on the aldehyde substrate present and some also have other functions unrelated to aldehyde oxidation. ALDH2 has by far the greatest affinity and reaction efficiency for AA (Ref 3) although ALDH1 B1 may also be involved in its metabolism (Ref 4). These two mitochondrial enzymes are expressed in numerous tissues of the body, but are most prevalent in the liver (Ref 4). ALDH deficiencies have been linked to the development of numerous diseases including Parkinson's disease (Ref 5), pyridoxine-dependent epilepsy (Ref 6), Sjögren-Larsson syndrome (Ref 6), as well as some which are also partially linked to the presence of AA, such as alcoholic liver disease (Refs 7,8), ethanol-induced cancers (Ref 6), ischaemic tissue diseases (Ref 9) and Alzheimer's disease (Ref 10). In spite of this, disulfiram (DSF; trade-name Antabuse), an ALDH inhibitor, has been prescribed as a treatment for alcohol abuse, causing greatly increased concentrations of AA in the blood and contributing to an extended “hangover” effect. The drug has also been proposed as a treatment for cocaine addiction (Ref 11). On the other hand, high ALDH expression has been associated with heightened metastatic potential in breast (Ref 12), prostate (Ref 13) and pancreatic (Ref 14) cancer stem cells in vitro and has also been shown to play a role in drug resistance (Refs 15,16). Furthermore, in vitro (Ref 17) and in vivo (ref 18) studies suggest that the aforementioned ALDH inhibitor, DSF, may contribute to future anticancer therapies. Heightened ALDH activity is also an important marker for some stem cells, such as haematopoietic stem cells.

DMSO is known to inhibit horse liver alcohol dehydrogenase, being competitive with aldehyde, and is assumed to compete for binding to the enzyme's carbonyl binding site (Science. 1968 Apr 19;160(3825):317-9).

### Analysis of ALDH Activity

In vitro analyses of ALDH activity are commonly performed on solutions containing ALDH, NAD+ and a suitable aldehyde substrate, by measuring the change in the absorbance of solutions at 340 nm, which provides an indication of the amount of NADH produced from the enzyme reactions (Refs 3, 4). This method of analysis has been widely used in the study of individual enzymes present in cell lysates, but is not useful for analysing metabolism in live cells, and cannot provide direct information on substrates. More recently, a flow cytometry-based assay has been developed, viz. ALDEFLUOR® (STEMCELL Technologies Inc.), for the selection of so-called ALDH^{br} (ALDH-bright) cells, including haematopoietic stem cells (Ref 19) from a mixed population, employing the ALDH inhibitor diethylaminobenzaldehyde (DEAB) as a control. This technique has also been used to identify differences in the levels of ALDH expression in a number of lung cancer cell lines, which, it is hypothesised, may be due to the stem cell-like properties of some cancer cell lines (Ref 20). It has also found utility in identifying and separating populations of cells based on their ALDH expression levels, but it is not designed for in vitro analyses of ALDH-mediated metabolism and enzyme kinetics.

Several gas/vapour phase mass spectrometry techniques have been used to analyse the headspaces of a number of cancer derived and non-cancerous primary and transformed human cell types cultured in vitro, with AA commonly observed. AA was seen to have been produced by human lung cancer cell lines CALU-1 and SK-MES, relative to their respective media, by Smith and co-workers, using selected ion flow tube mass spectrometry (SIFT-MS), a real-time trace gas analysis technique (Ref 21). Numerous similar studies have since shown the compound to be either produced (Refs 22-24) or consumed (Refs 22, 24-26) by various cell types. Noteworthy is the consistency of a headspace analysis study in which it was found that BEAS2B produced AA, and A549 removed AA from their respective media (Ref 24) with the previously mentioned ALDEFLUOR study in which it was found that these cell types contained very low (0.3%) and very high (94%) ALDH expression levels respectively (Ref 20). These results indicate that the production/consumption of AA from the headspace of a cell culture is correlated with the levels of ALDH expression and/or activity within the cells. This relationship is explored further in the present study using SIFT MS.

### Detection and Quantification of Volatile Compounds

Volatile compounds possess a low boiling point, consequently molecules of such compounds typically evaporate or sublimate from liquid or solid form to exist in gaseous form even at room temperature and pressure. Methods for the detection and quantification of volatile compounds are therefore often performed on the gaseous phase.

Techniques for detection and quantification of volatile compounds include those based on gas chromatography (GC) methodology—a technique which can be used to separate compounds for subsequent identification and quantification. Separation can be on the basis of, for example, boiling point, polarity, size or stereochemistry. Detection techniques can also be used in conjunction with gas chromatography for the detection of the separated compounds, e.g. gas chromatography-flame ionization detection (FID), gas chromatography-UV spectrometry (GC-UV), gas chromatography-pulsed flame photometric detection, thermal conductivity detector (TCD), nitrogen phosphorous detector (NPD), electron capture detector (ECD) or atomic emission detector (AED). ‘Electronic nose’ devices are also being developed that are capable of detecting and quantifying compounds in gas/vapour through use of electronic sensing (‘e-sensing’).

### Mass Spectrometry

Mass spectrometry is commonly used to detect and quantifying compounds from within a sample. In brief, samples are initially ionised, the resulting ions being separated by their charge to mass ratio and numbers detected. Compounds are recognised by their signature ion profile.

Several Mass Spectrometry-based techniques are suitable for detecting and quantifying volatile compounds, for example proton transfer mass spectrometer (PTR-MS) allows measurement of trace components with concentrations down to the parts-per-trillion by volume (pptv) level (International Journal of Mass Spectrometry and Ion Processes Volume 173, Issue 3, February 1998, Pages 191-241). Further examples include high-resolution electrospray ionization mass spectrometry (HR-ESI-MS) and secondary electrospray ionization-mass spectrometry (SESI-MS)—which has been used as a real-time clinical diagnostic tool in detecting volatile organic compounds (J. Clin. Microbiol. December 2010 vol. 48 no. 12 4426-4431).

Gas chromatography-mass spectrometry (GC-MS) based techniques have also been used, e.g. see J Sci Food Agric. 2011 May;91(7):1187-98. Several techniques for sample injection can be utilised in GC-MS, including: split, split-less, thermal desorption, headspace or solid phase micro extraction (SPME).

Selected Ion Flow Tube Mass Spectrometry (SIFT-MS)

Selected ion flow tube mass spectrometry (SIFT-MS) is an analytical technique for the real-time quantification of trace gases; utilising chemical ionization of the trace gas molecules in samples by positive precursor ions (typically H₃O+, NO+, and O₂+). Reactions between the precursor ions and trace gas molecules proceed for an accurately defined time, the precursor and product ions being detected and counted by a downstream mass spectrometer, allowing quantification. Absolute concentrations of trace gases in single breath exhalation can be determined by SIFT-MS down to parts-per-billion (ppb) levels without requirement for sample preparation or calibration with standards (see Eur. J. Mass. Spectrom. 13,77-82 (2007); Mass Spectrometry Reviews 2005 Volume 24, Issue 5, pages 661-700). Rapid reaction times in SIFT-MS (typically occurring in milliseconds) allow analysis to be conducted in real time. SIFT-MS differs from other chemical ionisation techniques such as PTR-MS through use of multiple reagent ion species in analysing a sample, enhancing identification (see Mass Spectrom Reviews 24 (2005) 661).

Applications for SIFT-MS include detection of breath metabolites indicative of disease, detection of compounds in exhaust gases, detection of volatile compounds in rumen gases and detection of volatile compounds in headspace of urine and cell cultures.

The methodology of SIFT-MS analyses for the sampling and quantification of compounds in the headspace of liquid samples has been used to detect volatile biomarkers emitted from lung cancer cell lines (Rapid Communic. Mass Spectrom. Vol 17, Issue 8, pages 845-850). SIFT-MS can be used to analyse metabolic processes occurring within cells in culture through analysis of any volatile metabolic compounds produced and present in the headspace of cell culture vessels.

### SUMMARY OF THE INVENTION

The inventors have identified dimethyl sulphide (DMS) as a marker of cell proliferation, in particular its production in cultured cells by reduction of dimethyl sulphoxide (DMSO).

In an aspect of the present invention the use of DMS as a marker of cell proliferation or cell death, or apoptosis, is provided.

In one aspect of the present invention a method for determining the number of cells in an in vitro culture of cells is provided, the method comprising measuring the DMS produced by the cultured cells.

In an aspect of the present invention a method of monitoring or measuring cell proliferation comprising detecting DMS produced by cells is provided.

In an aspect of the present invention an in vitro method of monitoring or measuring cell proliferation comprising detecting DMS produced by cells in vitro is provided.

In an aspect of the present invention an in vitro method of monitoring or measuring cell death comprising detecting DMS produced by cells in vitro is provided.

The cells are preferably cultured in the presence of DMSO or in the presence of a substrate capable of enzymatic conversion to DMS by an enzyme or enzymes present in the cells. The enzymatic conversion may be a reduction or molecular lysis/splitting/cleavage reaction.

In one aspect of the present invention a method for determining the number of cells in an in vitro culture of cells is provided, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, measuring the DMS produced by the cultured cells.

In an aspect of the present invention a method of determining a change in the number of cells contained in a cell culture is provided, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, measuring the DMS produced by the cultured cells at a first time point and determining the number of cells in the culture at said first time point, measuring the DMS produced by the cultured cells at a second time point and determining the number of cells in the culture at said second time point.

In another aspect of the present invention a method of determining the ability of a test compound or test condition to cause cell death of cells cultured in vitro is provided, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, contacting the cells with a test compound or subjecting the cells to a test condition, measuring the DMS produced by the cultured cells.

In a further aspect of the present invention a method of determining the ability of a test compound or test condition to enhance proliferation of cells cultured in vitro is provided, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce dimethyl sulphide DMS, contacting the cells with a test compound or subjecting the cells to a test condition, measuring the DMS produced by the cultured cells.

In some embodiments the cells are contacted with the test compound or subjected to the test condition after having been cultured for a period of time sufficient for the cells to produce DMS. The DMS produced by the cells may then be measured before and after contact of the cells with the test compound or before and after subjecting the cells to the test condition to determine a change in the level of DMS produced by the cultured cells, the change being indicative of reduction or increase in the number of live cells in the culture.

In some embodiments the cells are contacted with the test compound or subjected to the test condition during culture of the cells for a period of time sufficient for the cells to produce DMS. The DMS produced by the cells may then be measured and compared against a standard data set in order to determine if the test compound or test condition has reduced or increased the number of live cells in the culture.

In another aspect of the present invention a kit is provided comprising DMSO in a container and information indicating a plurality of DMS concentrations produced by a respective plurality of discrete numbers of cells per amount of DMSO added to the culture and per the time period of the culture with DMSO.

The information may take the form of one or more standard data sets, or standard curves. The information may be provided on a data carrier, such as a computer readable medium, e.g. diskette, memory stick, compact disc or other electronic data carrier.

### Description

The inventors investigated the activities of intracellular enzymes including the effects of the enzyme inhibitors DEAB and DSF on AA present in cultures of immortalised hepatocellular carcinoma cell line (hepG2) and a primary human bone marrow derived mesenchymal stem cell (hMSC). Using SIFT-MS, real time absolute quantification of volatile compounds present in the headspace of cell cultures at concentrations down to parts-per-billion by volume (ppbv) with no requirement for repeated calibration was performed.

During the course of these experiments, the inventors observed that the solvent used to dissolve the inhibitor compounds, dimethyl sulphoxide (DMSO), was reduced to volatile dimethyl sulphide (DMS) by both cell types. The inhibitory effects of DEAB and DSF on this reduction reaction were also studied using SIFT-MS to analyse the concentration of DMS in the gas/vapour phase above the cell cultures.

The inventors noted that when both cell types were culture in the presence of DMSO the concentration of DMS in the culture headspace was directly proportional to the number of cells in the culture, across a range of zero to 3×10⁷ cells (FIG. 4b). The concentration of DMS in the headspace was reduced when the inhibitor DEAB was included in the culture media (FIG. 4b).

As such, the inventors have realised that the level of DMS produced by cells cultured in DMSO and capable of reducing DMSO to DMS can be used to directly indicate the number of live cells present in the culture. This realisation provides the basis of an assay for determining the number of cells in a culture of cells, in particular the number of live cells in a culture, in which assay cells are cultured in the presence of DMSO and the concentration of DMS produced by the cultured cells is measured and used to determine the number of live cells in the culture.

The inventors have realised that an assay for determining the number of cells in culture, in particular the number of live cells in a culture, can be designed in which the cells are cultured in the presence of DMSO and the concentration of DMS produced by the cultured cells is measured and used to determine the number of live cells in the culture.

The present invention utilises the detection and quantification of DMS, produced by cells provided with DMSO, in the headspace of cell cultures at concentrations down to parts-per-billion by volume (ppbv), preferably in real-time, in a method of quantifying cell numbers when compared to DMS quantities derived from a series of known standards.

Quantification can be conducted on cells in situ, i.e. without removal from cell culture vessel of cell samples or without the need to detach cells from growth substrates.

As the enzymes responsible for the conversion of DMSO to DMS are present in diverse human tissue and mammalian tissue in general the invention can be widely used in quantifying cell numbers in mammalian cell culture. Furthermore, as the necessary enzymes are found in diverse organisms such including insects, yeast and bacteria it is expected that the invention can be practiced on cell cultures of cells derived from many organisms. Detecting the presence of such enzymes in cells can be achieved through use of, for example, polymerase chain reactions (PCRs). The detection of DMS by cells provided with DMSO can be conducted using, for example, mass spectrometry techniques.

Due to the sensitivity of the method, low levels of DMSO can be used with associated low toxicity—the invention can therefore be practiced on cells without the destruction of said cells. Suitability of cells for use with DMSO concentrations can be easily assayed, e.g. with use of live-dead cell staining. The invention can therefore be used multiple times on a population of cells, for example to determine growth kinetics over time or to determine when a desired quantity of cells is attained.

Use of a detector able to detect trace quantities of DMS in real time allows the method to be practiced on small cell quantities and cell cultures to be continuously monitored and stopped when desired quantities of cells are produced.

Methods according to the present invention involve measuring a DMS concentration produced by cultured cells, thereby indicating the number of cells in the culture. Methods according to the present invention may further comprise comparing a DMS concentration measured for a cell culture, or a change in DMS concentration measured for a cell culture over a given time period, with information contained in a standard data set.

Using Henry's Law and the Henry's Law coefficients, liquid-phase concentrations of DMS from the culture headspace can be calculated, which in turn can be used to calculate DMS production rates per cell for the particular conditions of the culture. These may be expressed as molecules/cell/min.

In methods of determining a change in the number of cells contained in a cell culture, measurements of DMS produced by the cells may be made at a plurality of time points, e.g. at one, two, three, four or five time points. Optionally, these may be evenly spaced time points, e.g. every 4, 8, 12, 16, 20, or 24 hours from the first contact of the cells with DMSO in the culture. At each time point the measured DMS can be used to determine a number of cells in the culture, and thereby a change in the number of cells in the culture over time can be determined.

In any method described herein, DMS concentration may be measured at the start of the culture period, e.g. when DMSO is added to the culture media, in order to establish a background level of DMS which may be subtracted from DMS measurements taken during the culture period.

By contacting the cells with a test compound or placing them under a test condition the effect of the test compound or test condition may be determined on the growth of the cells being cultured, e.g. to determine if it reduces/slows the rate of growth or increases it. This may also indicate if the test compound or test condition is capable of causing, inducing or facilitating cell death, apoptosis, cell division or cell proliferation.

A standard data set may contain information (e.g. in the form of one or a plurality of tables, spread sheets and/or charts) indicating a plurality of DMS concentrations produced by a respective plurality of discrete numbers of cells per amount of DMSO added to the culture and per the time period of the culture with DMSO.

That is, the information may describe the concentration of DMS produced by a given number of cells of a certain type having been cultured in a given amount of DMSO for a given amount of time under given culture conditions (for example, the concentrations of DMS produced by 10⁴, 10⁵, 10⁶, and 10⁷ human MSCs each cultured for 16 hours at 37° C. in DMEM containing 0.1% v/v DMSO).

In some embodiments, standard data sets are created by measurement of DMS produced in the headspace of cell cultures from known quantities of cells of a given type, cultured under defined culture conditions and for a defined period of time. Multiple independent replicates of such measurements may be made. For example, an ascending sequence of cell numbers may be used. For example, a series of cell numbers (×10⁶) starting at 1, 5, 10, 15, 20, 25, 30 and continuing for multiple further iterations of increasing cell numbers may be used to produce a standard data set.

Thus, in methods according to the present invention, the number of cells in the test culture, or change (increase or decrease) in the number of cells in the culture can be determined by referring to the appropriate standard data set. In doing so, it may be appropriate to plot or tabulate the relationship between cell quantity and DMS produced by the cell culture.

Where a change in DMS production is being measured, at least two measurements of DMS concentration will normally be taken during the course of the cell culture, e.g. at 16 hours and then at 32 hours. A reduction in DMS produced over time is indicative of cell death in the cell culture, i.e. reduction of the number of cells contained in the culture. An increase in DMS production over time is indicative of cell proliferation in the cell culture, i.e. expansion of the number of cells contained in the culture. Measuring the rate of change of DMS production can be used to indicate the rate of cell death or cell proliferation. By measuring the change in DMS production in response to a test compound(s) or test condition(s) added to the cell culture, the effect of test compounds or test conditions on cell death or apoptosis, on rate of cell death or apoptosis, on cell proliferation or on rate of cell proliferation can be investigated. Such methods provide for the screening of test compounds or test conditions for their activity in inducing or enhancing cell death, apoptosis or cell proliferation.

Cell death or apoptosis may be separately confirmed using standard assay techniques. Cell death assays include an MTT assay (e.g. using the Promega CellTiter 96® AQueous One Solution Cell Proliferation Assay (MTS)), Trypan Blue staining, Invitrogen's LIVE/DEAD Viability/Cytotoxicity Kit for Animal Cells. Apoptosis assays include the Terminal deoxynucleotidyl transferase dUTP nick end labeling (TUNEL) assay, Promega's Apo-ONE® Homogeneous Caspase-3/7 Assay, or Life Technologies' Multiparametric Apoptosis Assays for Flow Cytometry.

The inventors have shown that by measuring DMS produced by a cell culture, e.g. DMS concentration in the culture headspace, the number of cells in the culture can be determined.

The number of cells determined is expected to be representative of the cells capable of reducing DMSO to DMS, which is expected to at least provide an approximation of the number of live or viable cells, i.e. excluding dead cells, particularly dead cells no longer capable of enzymatic reduction of DMSO to DMS.

The determination of the number of cells in the culture may be an estimate or approximation to the nearest 10, 100, 1000 or 10,000, and will typically be at least to the nearest order of 10 magnitude (i.e. 10^{x} where x=1, 2, 3, 4, 5, 6, 7, 8 etc.) or less, and typically to the nearest integer of an order of magnitude, e.g. Y×10x where Y=1, 2, 3, 4, 5, 6, 7, 8, or 9, and x=1, 2, 3, 4, 5, 6, 7, 8 etc.).

### Methionine Sulfoxide Reductases (MSRs)

Methionine sulfoxide reductases (MSRs) are a family of enzymes that catalyse the reduction of free and protein bound methionine sulfoxides and are found in virtually all organisms.

MSR functions have been suggested to include regulating protein function through oxidation/reduction of methionine residues in proteins and repairing oxidative damage. Two distinct classes of these enzymes exist, MsrA and MsrB, which selectively reduce the two methionine sulfoxide epimers, methionine-S-sulfoxide and methionine-R-sulfoxide, respectively. An assay for the enzymatic activity of MsrA and MsrB is described in Arch Biochem Biophys. 2012 Nov. 1;527(1):1-5.

Homologs of both MsrA and MsrB genes have been identified in most living organisms, including mammals, plants, yeast, nematodes, fruit flies and prokaryotes (see Molecular Biology of the Cell Vol. 15, 1055-1064, March 2004; Biochim Biophys Acta. 2005 Jan. 17;1703(2):221-9). In general, mammals possess one gene encoding MsrA and at least three genes encoding MsrBs. Humans possess threes MsrB genes: MsrB1 (selenoprotein R/ Selenoprotein X), MsrB2 (CBS-1) and MsrB3. MsrB1 is identified in Journal of Biological Chemistry, 274, 38147-38154; MsrB2 in Gene Volume 233, Issues 1-2, 11 Jun. 1999, Pages 233-240 and MsrB3 in Biochem Biophys Res Commun. 2012 Mar. 2;419(1):20-6. Techniques to detect expression of hMsrB1-3 are described in Invest Ophthalmol Vis Sci. 2005 June;46(6):2107-12.

Multiple splice forms of the MSR genes occur, e.g. MsrB3A and MsrB3B, and the protein products of these splice variants can exhibit differential sub-cellular localisation, for example to the cytosol, nucleus, endoplasmic reticulum and/or mitochondria (see Biochimica et Biophysica Acta (BBA)—Proteins and Proteomics Vol. 1703, Issue 2, Pages 239-247 and Molecular Biology of the Cell Vol. 15, 1055-1064, March 2004).

### Methionine Sulfoxide Reductase A (MsrA)

DMSO can be reduced by the actions of the enzyme methionine sulfoxide reductase A (MsrA). MsrA can use as a substrate a protein containing Met(O) and other organic compounds which contain an alkyl sulfoxide group (Proc. Natl. Acad. Sci. USA Vol. 93, pp. 2095-2099; BMB Rep., 2009,42, 580-585).

The cloning of human MsrA (hMsrA), expression data and assay for activity is disclosed in FEBS Letter Volume 456, Issue 1, 30 Jul. 1999, Pages 17-21. Mechanism of activity is described in PNAS 2011 vol. 108 no. 26 10472-10477.

MsrA enzymes have been detected in diverse animal tissues and organisms, including bacteria (e.g. see J Bacteriol. 2005 August; 187(16): 5831-5836) and yeast (e.g. see Proc Natl Acad Sci U S A. 2004 May 25; 101(21): 7999-8004). The polypeptide sequence and function is highly conserved. Human MsrA (hMsrA) is widely expressed in different tissue types (FEBS Letters Vol. 456, Issue 1, Pages 17-21, 1999).

Mammalian MsrA occurs in multiple alternatively spliced forms. These encode isoforms of the MsrA polypeptide, some of which contain an N-terminal mitochondrial signal peptide and are distributed between mitochondria and cytosol. Further isoforms utilise an alternative first exon or alternative first exon splicing. The differential presence of targeting signals in alternative forms can cause differential sub-cellular localisation of isoform variants.

### Dimethyl Sulphide (DMS)

Dimethyl sulphide (DMS) has the formula (CH₃)₂S and is produced by the reduction of dimethyl sulfoxide (DMSO; (CH₃)₂SO). DMS is a volatile compound and can be detected using gas chromatography or GC-MS based methods, biosensors (e.g. see Applied Microbiology and Biotechnology April 2006, Volume 70, Issue 4, pp 397-402), flame photometric detector (e.g. see Marine Chemistry Volume 14, Issue 3, February 1984, Pages 267-279), or SIFT-MS.

DMSO is a commonly used laboratory reagent, for example as a solvent for dissolving compounds administered to cell cultures or as a cryoprotectant. Use of DMSO concentrations of up to 10% (v/v) caused no observable cytotoxicity in Caco2/TC7 colon tumour cell cultures (Biol Pharm Bull. 2002 December;25(12):1600-3). Solutions of 5-10% (v/v) DMSO solutions are used in protocols for the freezing of cells (including bacterial and mammalian).

In the present invention, cells may be cultured in medium containing DMSO. The concentration of DMSO in the culture medium may be selected to suit the type of cells being cultured and aim of the culture method. Suitable DMSO concentrations include one or more of 0.001% v/v to 20%, 0.01% to 10%, 0.01% to 9%, 0.01% to 8%, 0.01% to 7%, 0.01% to 6%, 0.01% to 5%, 0.01% to 4%, 0.01% to 3%, 0.01% to 2%, 0.01% to 1%, 0.1% to 10%, 0.1% to 9%, 0.1% to 8%, 0.1% to 7%, 0.1% to 6%, 0.1% to 5%, 0.1% to 4%, 0.1% to 3%, 0.1% to 2%, 0.1% to 1%, 0.01% to 1%, 0.02% to 1%, 0.03% to 1%, 0.04% to 1%, 0.05% to 1%, 0.06% to 1%, 0.07% to 1%, 0.08% to 1%, 0.09% to 1%, 0.2% to 1%, 0.3% to 1%, 0.4% to 1%, 0.5% to 1%, 0.6% to 1%, 0.7% to 1%, 0.8% to 1%, 0.9% to 1%, or one or more of 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 8% or 10%.

Alternatively, suitable DMSO concentrations can be one or more of 145 μM to 2.9 M, 1.450 mM to 1.45 M, 1.45 mM to 1.305 M, 1.45 mM to 1.16 M, 1.45 mM to 1.015 M, 1.45 mM to 870 mM, 1.45 mM to 725 mM, 1.45 mM to 580 mM, 1.45 mM to 435 mM, 1.45 mM to 290 mM, 1.45 mM to 145 mM, 14.5 mM to 1.450 M, 14.5 mM to 1.305 M, 14.5 mM to 1.16 M, 14.5 mM to 1.015 M, 14.5 mM to 870 mM, 14.5 M to 725 mM, 14.5 mM to 580 mM, 14.5 mM to 435 mM, 14.5 mM to 290 mM, 14.5 mM to 145 mM, 1.45 mM to 145 mM, 2.9 mM to 145 mM, 4.35 mM to 145 mM, 5.8 mM to 145 mM, 7.25 mM to 145 mM, 8.7 mM to 145 mM, 10.15 mM to 145 mM, 11.6 mM to 145 mM, 13.05 mM to 145 mM, 29 mM to 145 mM, 43.5 mM to 145 mM, 58 mM to 145 mM, 72.5 mM to 145 mM, 87 mM to 145 mM, 101.5 mM to 145 mM, 116 mM to 145 mM, 130.5 mM to 145 mM, or one or more of 1.45 mM, 2.9 mM, 4.35 mM, 5.8 mM, 7.25 mM, 8.7 mM, 10.15 mM, 11.6 mM, 13.05 mM, 14.5 mM, 29 mM, 43.5 mM, 58 mM, 72.5 mM, 87 mM, 101.5 mM, 116 mM, 130.5 mM, 145 mM, 290 mM, 435 mM, 580 mM, 725 mM, 870 mM, 1,015 mM, 1,160 mM, 1305 mM or 1450 mM.

Measurement of DMS may be conducted by a number of known techniques, including Mass Spectrometry, e.g. SIFT-MS, gas chromatography, or gas chromatography- mass spectrometry (GC-MS). In one embodiment, DMS is detected and quantified by mass spectrometry. In preferred embodiments, the mass spectrometry method is SIFT-MS.

### Cells

Cells may be of any kind provided they are capable of reducing DMSO to DMS, or are capable of producing DMS when cultured in the presence of a substrate capable of enzymatic conversion to DMS by an enzyme or enzymes present in the cells.

The cells may be eukaryotic or prokaryotic. The cells may be:
non-human cells, e.g. rabbit, guinea pig, rat, mouse or other rodent (including cells from any animal in the order Rodentia), cat, dog, pig, sheep, goat, cattle, horse, non-human primate or other non-human vertebrate organism; and/or
non-human mammalian cells; and/or
human cells; and/or
bacterial, fungal, plant, or algae.

The cells may have been genetically manipulated to produce recombinant gene products such as proteins or antibiotics.

Cells may be somatic cells, adult cells, immortalised cells, cancer/tumor cells or stem cells and may, for example, be obtained from established cell lines or from patient biopsy.

Examples of suitable cells include mesenchymal stem cells (MSCs), hepG2 cells and MG-63 cells.

Cells are preferably capable of converting DMSO to DMS. The ability of cells to produce DMS can be assayed by culturing cells in growth media containing DMSO, for example 0.1% v/v, under suitable growth conditions, for example 37° C., for around 16 hours and measuring DMS levels in the cell culture headspace using SIFT-MS. Suitable cell growth conditions can be easily determined, for example by constructing a growth curve (in which measurement of cell numbers is plotted against time). Cell numbers can be measured using, for example, using a haemocytometer.

Cells may express an enzyme capable of converting DMSO to DMS, e.g. one or both of an aldehyde dehydrogenase (ALDH) and a methionine sulfoxide reductase (Msr), e.g. MsrA. In some embodiments, the cells comprise one or more methionine sulfoxide reductase enzymes. In some embodiments, the cells comprise MsrA.

Stem cells may be stem cells of any kind.

Stem cells may be pluripotent, e.g. embryonic stem cells (ESC) or human embryonic stem cells (hESC), or induced pluripotent stem cells. Pluripotency may be determined by use of suitable assays. Such assays may comprise detecting one or more markers of pluripotency, e.g. SSEA-1 antigen, alkaline phosphatase activity, detection of Oct-4 gene and/or protein expression, by observing the extent of teratoma formation in SCID mice or formation of embryoid bodies. The pluripotency of hESC may be defined by the expression of markers such as Oct-4, SSEA-4, Tra-1-60, Tra-1-81, SOX-2 and GCTM-2.

Stem cells may be adult stem cells and/or multipotent stem cells.

Adult stem cells comprise a wide variety of types including neuronal, skin and the blood forming stem cells which are the active component in bone marrow transplantation. These latter stem cell types are also the principal feature of umbilical cord-derived stem cells. Adult stem cells can mature both in the laboratory and in the body into functional, more specialised cell types although the exact number of cell types is limited by the type of stem cell chosen.

Multipotent stem cells are true stem cells but can only differentiate into a limited number of types. For example, the bone marrow contains multipotent stem cells that give rise to all the cells of the blood but not to other types of cells. Multipotent stem cells are found in adult animals. It is thought that every organ in the body (brain, liver) contains them where they can replace dead or damaged cells.

Examples of adult/multipotent stem cells include hematopoietic stem cells, neural stem cells or mesenchymal stem cells. Adult mesenchymal stem cells are capable of differentiation into connective tissue and/or bone cells such as chondrocytes, osteoblasts, myocytes and adipocytes.

Methods of characterising stem cells are known in the art, and include the use of standard assay methods such as clonal assay, flow cytometry, long-term culture and molecular biological techniques e.g. PCR, RT-PCR and Southern blotting.

In addition to morphological differences, human and murine pluripotent stem cells differ in their expression of a number of cell surface antigens such as Oct4, SSEA-1, SSEA-4, Tra-1-60, and Tra-1-81, Flk-1, Tie-2 and c-kit.

Stem cells cultured in the present invention may be obtained or derived from existing cultures or directly from any adult, embryonic or fetal tissue, including blood, bone marrow, skin, epithelia or umbilical cord (a tissue that is normally discarded).

Optionally, the stem cells are not human embryonic stem cells and/or are not obtained by destruction of a human embryo. Human embryonic stem cells may be obtained from established cell lines, e.g. as available from ATCC.

### Cell Culture

Cell culture refers to the growth of cells outside their natural environment, typically in vitro. Mammalian cells are typically grown in suspension (i.e. free floating in the culture medium) or as adherent cells (i.e. on an artificial substrate). A large proportion of cells derived from vertebrates are anchorage-dependent requiring culture on a suitable substrate, for example tissue culture plastic of microcarriers. Detachment of such cells, for example temporarily for splitting cell cultures or in cell quantification assays, can often be accomplished using transient treatment with trypsin enzyme.

Non-mammalian cells can also be grown as a cell suspension in a liquid medium or on a solid medium; examples being callus cultures for plant cells or bacteria and yeast cells grown on gels such as agar. Cells may also grow as aggregates, for example as biofilms.

The headspace is the unfilled space above the contents of a closed container. With regard to cell culture, the headspace is the volume of a cell culture vessel unfilled with culture medium. Where measurement of volatile compounds present in the headspace is undertaken the culture vessel will typically be sealed to allow volatile compounds to accumulate in the headspace and to prevent mixing with gases from the external environment.

In some embodiments cells are cultured in sealed cell culture vessels, e.g. culture dishes, flasks or bottles. In some embodiments cells are cultured in suspension or as an adherent layer.

In some embodiments the cells may be cultured in a bioreactor or fermenter suitable for the large scale production of cellular products, such as antibiotics, proteins, polypeptides or peptides (optionally recombinant proteins, polypeptides or peptides).

In some embodiments DMS production is measured in cell cultures near confluence or at/post-confluence. In some other embodiments DMS production may be measured in log growth phase before they reach confluence (contact inhibition). The time taken for cells to reach confluence varies, dependent on the cell division rate of the cell line and also the initial seeding density of cells.

Cells may be cultured for a period of time sufficient for the cells to produce DMS following addition of DMSO to the culture. This time period may vary with the type of cells being cultured. For example, it may be any of 1, 2, 3, 4, 5, 6, 7, 8, 9, 10, 11, 12, 13, 14, 15, 16, 17, 18, 19, 20, 21, 22, 23 or 24 hours, or any one of 1, 2, 3, 4, 5, 6, or 7 days. Where a change in DMS concentration is being measured, individual DMS concentration measurements may be made once every 1, 2, 3, 4, 5, 6, 7, 8, 9, 10, 11, 12, 13, 14, 15, 16, 17, 18, 19, 20, 21, 22, 23 or 24 hours, or every 1, 2, 3, 4, 5, 6, or 7 days.

Cells may be cultured at a temperature appropriate for a given cell type, e.g. 37° C. for mammalian cells. When comparing DMS production at different time points or with a standard data set the same culture temperature will typically be maintained between measurements or between the culture and standard data set.

Cells will typically be cultured at atmospheric pressure. In some cultures, the gaseous environment of the culture may be artificially maintained according to known optimum culture conditions for the cell type, e.g. to contain 5% CO₂ when culturing some mammalian cells.

### Cell Culture Substrates

Cell culture substrates may be two dimensional or three dimensional.

Two dimensional cell culture substrates include plastic or glass surfaces, sheets or layers and may be provided in the form of culture dishes, bottles or flasks.

Two and three dimensional substrates may have a proteinaceous or polymer surface or coating, e.g. comprising proteins or polymers such as collagen, fibronectin, laminin, fibronectin, laminin, entactin, Matrigel™, poly-L-Lysine, or poly-L-Ornithine.

In some embodiments the substrate may be a three dimensional scaffold, and may be made of a material onto which the cells may be adhered or in which the cells may be impregnated. The material can be seeded with the selected cells. As such, the material may provide a scaffold or matrix support. The material may be suitable for implantation in tissue, or may be suitable for administration to the body (e.g. as microcapsules in solution).

Three dimensional cell culture scaffolds allow for the culture of cells in order to grow artificial tissues having defined three-dimensional shape, thus being useful for the production of engineered tissue constructs.

In some embodiments the material should be biocompatible, i.e. non-toxic and of low immunogenicity (most preferably non-immunogenic). The material may be biodegradable.

Suitable materials may be soft and/or flexible, e.g. hydrogels, fibrin web or mesh, or collagen sponges. A “hydrogel” is a substance formed when an organic polymer, which can be natural or synthetic, is set or solidified to create a three-dimensional open-lattice structure that entraps molecules of water or other solutions to form a gel. Solidification can occur by aggregation, coagulation, hydrophobic interactions or cross-linking.

Alternatively suitable materials may be relatively rigid structures, e.g. formed from solid materials such as plastics or biologically inert metals such as titanium.

The material may have a porous matrix structure which may be provided by a cross-linked polymer. The scaffold/matrix is preferably permeable to nutrients and growth factors required for bone growth.

Matrix structures may be formed by crosslinking fibres, e.g. fibrin or collagen, or of liquid films of sodium alginate, chitosan, or other polysaccharides with suitable crosslinkers, e.g.

calcium salts, polyacrylic acid, heparin. Alternatively scaffolds may be formed as a gel, fabricated by collagen or alginates, crosslinked using well established methods known to those skilled in the art.

Suitable polymer materials for matrix formation include biodegradable/bioresorbable polymers which may be chosen from the group of: collagen, fibrin, chitosan, polycaprolactone, poly(DL-lactide-co-caprolactone), poly(L-lactide-co-caprolactone-co-glycolide), polyglycolide, polylactide, polyhydroxyalcanoates, co-polymers thereof, or non-biodegradable polymers which may be chosen from the group of: cellulose acetate; cellulose butyrate, alginate, agarose, polysulfone, polyurethane, polyacrylonitrile, sulfonated polysulfone, polyamide, polyacrylonitrile, polymethylmethacrylate, co-polymers thereof.

Collagen is a promising material for matrix construction owing to its biocompatibility and favourable property of supporting cell attachment and function (U.S. Pat. No. 5,019,087; Tanaka, S.; Takigawa, T.; Ichihara, S. & Nakamura, T. Mechanical properties of the bioabsorbable polyglycolic acid-collagen nerve guide tube Polymer Engineering & Science 2006, 46, 1461-1467). Collagen sponges are well known in the art (e.g. from Integra Life Sciences).

Fibrin scaffolds provide an alternative matrix material.

Suitable matrix structures also include de-cellularised human/mammalian matrices, and xenobiotic matrices which have been re-cellularised with the cells being cultured. It is difficult to measure cell number on these matrices using conventional means and so the methods of the present invention provide a particular advantage in this area.

### Quantification of Cell Number

Quantifying cell numbers (or cell counting) can be conducted by a number of techniques, including use of:
a counting chamber (or haemocytometer) in which a representative sample of cells in solution (usually diluted) is counted by eye against a grid,
cell plating, in which a representative sample of cells in solution (usually diluted) is plated on solid growth media and resulting single colonies counted,
spectrophotometry, in which cell numbers are calculated from the level of light absorbed by a sample of cells in solution,
electrical resistance, in which a Coulter counter is used to counts cells in a representative sample of cells in solution, or
flow cytometry, which detects cells in a representative sample of cells in solution.

### Kits

In some aspects of the present invention a kit of parts is provided. The kit may be an assay kit. In some embodiments the kit may have at least one container having a predetermined quantity of DMSO.

The DMSO may be provided as isolated DMSO or pre-mixed with other culture media components, optionally as a fully pre-mixed culture media. The pre-mix may be in ready to use format, e.g. a ready to use liquid/fluid/gel culture media formulation, or may be in a pre-use format such as a liquid/fluid or dried powder ready to be combined with other agents, e.g. dissolved in suitable solvent and/or or pre-mixed with other culture media components in order to provide a useable culture media.

The kit may contain items useful for cell culture, e.g. cell culture substrates such as plastic or glass sheets, layers, culture dishes, bottles or flasks, and/or proteinaceous or polymer substrates such as collagen, fibronectin, laminin, Matrigel™, poly-L-Lysine, or poly-L-Ornithine, and/or plastic or glass sheets, layers, culture dishes, bottles or flasks coated in one or more of such proteinaceous or polymer substrates, or other substrates, scaffolds or matrices described herein.

The kit may contain other items useful as cell culture media components such as DMEM, FBS, BSA, antibiotic(s) (e.g. penicillin, and/or streptomycin), amino acids, electrolytes, which may be provided in one or more additional containers.

The kit may include one or more standard data sets (e.g. in the form of one or a plurality of tables, spread sheets and/or charts) allowing for the comparison of a measured DMS concentration in order to determine the number of cells in the culture. The data sets, or standard curves may be provided as printed information, e.g. on paper, but may additionally or alternatively be provided on a computer readable medium. The computer readable medium may be a diskette, memory device, memory stick or card, compact disc or other electronic data carrier. The computer readable medium may contain the data sets in the form of one or a plurality of tables, spread sheets and/or charts, and may contain software allowing the user to access and manipulate the data sets, and enter recorded DMS concentrations and cell culture information (such as culture conditions (e.g. duration of culture, culture temperature, culture pressure, type of cell culture media) type of cells, concentration of DMSO in the culture media) to allow a comparison with a standard data set. The software may be executable to compare the input data with one or more of the standard data sets to provide an indication of the number of cells in the culture.

The kit may further comprise instructions for one or more of (i) the performance of cell culture, (ii) measurement of DMS concentration, and (iii) comparison of data obtained from the cell culture with one or more standard data sets to determine the number of cells in the cell culture.

### In Vitro Methods

Methods according to the present invention may be performed in vitro. The term “in vitro” is intended to encompass experiments with materials, biological substances, cells and/or tissues in laboratory conditions or in culture. Where the method is performed in vitro it may comprise an assay. In some instances the assay may be screening assay. Test compounds used in the screening assay may be obtained from a synthetic combinatorial peptide library, or may be synthetic peptides or peptide mimetic molecules. Other test compounds may comprise defined chemical entities, oligonucleotides or nucleic acid ligands.

### Test Compounds

Candidate test compounds may comprise any kind of compound, e.g. small molecule chemical entities (synthetic or naturally occurring) or biological agents such as antibodies and antibody products (e.g. monoclonal and polyclonal antibodies, single chain antibodies, chimeric antibodies and CDR-grated antibodies), peptides, polypeptides, proteins (e.g. growth factors) and nucleic acids (e.g. DNA or RNA).

Cell cultures may be contacted with one or more test compounds to determine the effect a compound has on the number of cells in the culture. This may be indicative of the effect of a test compound on cell death, apoptosis or cell proliferation.

In some instances the effect of a test compound may be to enhance or induce cell death or apoptosis. Cell death can be confirmed using standard assay techniques, e.g. Trypan Blue staining. Apoptosis can be determined by one of a number of techniques known to the person skilled in the art, e.g. the observing of morphological changes such as cytoplasmic blebbing, cell shrinkage, internucleosomal fragmentation and chromatin condensation. DNA cleavage typical of the apoptotic process may be demonstrated using TUNEL and DNA ladder assays.

### Test Conditions

Test conditions include selected cell culture environments, e.g. temperature, pressure, gaseous environment, partial pressure of a gas adjacent the cell culture or in the culture container, cell substrate, which may be varied to assess the effect on the number of cells in the cell culture.

Cell cultures may be subjected to one or more test conditions to determine the effect a particular condition has on the number of cells in the culture. This may be indicative of the effect of a test condition on cell death, apoptosis or cell proliferation.

In some instances the effect of a test condition may be to enhance or induce cell death or apoptosis. Cell death can be confirmed using standard assay techniques, e.g. Trypan Blue staining. Apoptosis can be determined by one of a number of techniques known to the person skilled in the art, e.g. the observing of morphological changes such as cytoplasmic blebbing, cell shrinkage, internucleosomal fragmentation and chromatin condensation. DNA cleavage typical

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1. Table showing SIFT-MS measurements of the headspace concentrations of acetone, ethanol, methanol, AA and DMS measured in non-treated (NT) DMEM medium, as well as medium containing 0.1% v/v DMSO (141 000 μM) alone, and with dissolved diethylaminobenzaldehyde (DEAB; 2001.1M) or disulfiram (DSF; 20 μM), following 16 hours sealed incubation with no cells present. All headspace concentrations are given in parts-per-billion by volume (ppbv). The instrumental error is typically within 10% (see Smith and Spanel, 2011 (ref. 32)), except in the case of DMS, which was present at concentrations which are approaching the limit of detection of the SIFT-MS instrument

FIG. 2. Graphs showing the AA concentrations, given in parts-per-billion by volume (ppbv), measured in the headspaces hepG2 cell cultures, against the cell number, following a 16-hour incubation period at 37° C. The cells were contained in 15 mL volumes of DMEM medium, inside sealed 150 mL glass bottles for the duration of this period. In (a), the cells were not treated with ALDH inhibitors (open circles), and the dashed line displays the results of a mathematical model, based on Michaelis-Menton enzyme kinetics. In (b), the cells were treated with 200 μM DEAB (closed circles) or 20 μM DSF (open squares) for 16 hours prior to the analysis. All experiments were performed in duplicate and the bars indicate the range of the obtained values. Note the change of scale of the y-axes.

FIG. 3. Graphs showing the measured headspace concentrations of AA (a) and DMS (b), in parts-per-billion by volume (ppbv), against the liquid phase DEAB concentration in cultures containing 1.5(10⁷) hepG2 cells in 15 mL of DMEM medium. The DEAB was dissolved in 0.1% v/v DMSO in all experiments. In samples of the medium alone, with no cells, no DMSO and no inhibitor compounds present, the AA concentration was measured to be 130 ppbv, as is indicated with the labelled, short-dashed line, and the measured DMS concentration was 10 ppbv. The long dashed curve is an “eye ball” variation following the experimental points.

FIG. 4. Graphs showing the headspace DMS concentrations against the numbers of hepG2 cells in DMEM media containing 0.1% v/v DMSO (open circles), 200 μM DEAB (closed circles) and 20 μM DSF (open squares). The results were obtained from two independent experiments, which, for clarity, are presented on 2 separate plots by the number of cells present as follows: (a) 1(10⁴) to 5(10⁶) cells; and (b) 0 to 3(10⁷) cells. Note that the x-axis in (a) is presented on a logarithmic scale, whereas in (b) the scale is linear. The concentration of DMS measured in the headspace of the DMEM medium alone was approximately 10 ppbv in both situations.

FIG. 5. Table showing AA and DMS concentrations measured in the headspace of hMSCs cultures (in glass bottles sealed by septa) containing typically 5 million cells in 10 mL of DMEM, following 24 hours pre-treatment and a further 16 hours of sealed incubation at 37° C. in the presence of DEAB or DSF. All samples contained 0.1% v/v DMSO (14 500 μM) with the exception of the non-treated cell-containing sample (NT). The mean headspace concentrations of acetaldehyde and DMS in medium without any cells were 89±37 and 6±4 ppbv respectively

FIG. 6. Microscopy images of hepG2 cells (a) prior to and (b-d) following overnight incubation inside sealed glass bottles. The cells in (a) and (b) were not treated with ALDH inhibitors, whereas in (c) and (d) 200 μM DEAB and 20 μM DSF were added to the contained medium respectively. Charts showing the results of ATPLite assays conducted on hepG2 (e); and hMSC (f), are also shown. The results were obtained following culture under normal conditions (non-treated; NT) or following 16 hours of treatment with 0.1% v/v DMSO, or with ALDH inhibitors: DEAB or DSF. The inhibitor concentrations are indicated on the x-axis where appropriate. The data is presented as the mean±standard error (N=8).

FIG. 7 Schematic illustration of the SIFT-MS instrument. The general ion chemistry occurring between the precursor ion H3O+ and reactant trace gas molecules M is also indicated.

FIG. 8. Chart showing time release of DMS following prolonged incubation of 5, 10 and 20 million MG-63 cells attached to glass with a medium containing 0.1% DMSO.

FIG. 9. Charts showing time release of DMS by varying numbers of MG63 following prolonged culture in collagen hydrogels and a medium containing 0.1% DMSO. (a) DMS concentration vs. incubation period. (b) DMS concentration vs. starting cell number.

FIG. 10. Charts showing time release of DMS by varying numbers of HEPG2 following prolonged culture in collagen hydrogels and a medium containing 0.1% DMSO. (a) DMS concentration vs. incubation period. (b) DMS concentration vs. starting cell number.

FIG. 11. (a) Partial illustration of SIFT-MS apparatus. SIFT-MS was used to determine the production of DMS by cells of MG-63 cell lines. (b) Chart showing the emissions of DMS from DMSO into the vapour phase. These were shown to be linearly related to the number of cells seeded to a collagen scaffold.

FIG. 12. Chart showing comparison of DMS production by HepG2 cells with WST-8 absorbance. Blue diamonds=WST-8 [upper diamond at x-axis=0, 5, 10 and lower diamond at x-axis=20]. Red diamonds=DMS [lower diamond at x-axis=0, 5, 10 and upper diamond at x-axis=20].

### EXAMPLE 1

### Materials and methods

### Cell Cultures

Human mesenchymal stem cells, hMSCs, are a primary cell type, and were isolated from a bone marrow aspirate sample (27 years old male; Lonza, US) using the plastic-adherence methodology and the hepG2 cells are of a human hepatocellular carcinoma cell line (Eton Bioscience, US), a cell line commonly used for the study of liver function. In both cases, the cells were cultured to confluence in Dulbecco's modified Eagle's medium (DMEM; Lonza, UK), supplemented with 10% v/v foetal bovine serum (FBS; Lonza, UK), 50 U mL⁻¹ penicillin-streptomycin (Lonza, UK) and 2 mM L-glutamine (Lonza, UK). The hMSCs additionally contained 1% v/v non-essential amino acids. The hMSCs were not cultured beyond passage number 4, whereas the well-differentiated hepG2 cells were analysed before passage number 20.

### Sample Preparation

The ALDH inhibitors DEAB (Sigma, UK) and disulfiram (DSF; Sigma, UK) were dissolved in DMSO (Sigma, UK). DMSO was selected to be the common solvent for the inhibition experiments, because it has a relatively low vapour pressure (1.8 mbar at 37° C.) and thus was not expected to interfere significantly with headspace analyses (further explanation below). The inhibitor solutions were then added to volumes of medium so that the final DMSO concentration was always 0.1% v/v (or ˜14 500 μM). In some experiments, inhibitors were added to hMSCs during routine culture, some 24 hours prior to proceeding to the next phase of the analysis, but this pre-treatment was not employed in the hepG2 experiments. It is important to note that maximum concentration of DSF in a DMSO solution is 20 mM, which imposed a limit on the maximum concentration in the cultures (0.1% v/v) of 20 μM. The most obvious alternative solvent to DMSO would have been ethanol, but a concentration of 0.1% v/v ethanol in the liquid phase would equate to a vapour phase concentration of around 200 ppmv at 37° C., assuming a Henry's Law coefficient of 71 mol kg⁻¹bar⁻¹, and this would have seriously interfered with the SIFT-MS measurements because of the ability of ethanol molecules at this concentration to totally deplete the precursor ions on which SIFTMS analyses depend. The importance of precursor ions in the analysis is explained further in the SIFT-MS section below. The method used for the preparation of cell cultures for SIFTMS headspace analysis was adapted from the previous studies published in 2003 (ref. 21) and 2009 (ref. 22). On the evening prior to the analysis, the cells were removed from the tissue culture flasks using trypsin, counted using a haemocytometer, and suspended in fresh DMEM media containing the ALDH inhibitor where appropriate, inside 150 mL glass bottles. HEPES buffer (Sigma, UK) was added to each cell suspension/media volume to a final concentration of 10 mM in order to ensure that the pH of the sample did not change significantly overnight. The headspace of each bottle was purged with dry cylinder air before the bottles were finally sealed with metal caps, which incorporate rubber septa, and placed inside an incubator to be held at 37° C. for around 16 hours/overnight.

### SIFT-MS Analysis

The methodology of SIFT-MS analyses for the sampling and quantification of compounds in the headspace of liquid samples has been described in detail previously (Refs 21,22,27-32). Briefly, the contained headspace of a sealed 150 mL glass bottle, held at 37° C., is sampled via a hypodermic needle, which penetrates a septum at the bottle cap, allowing the gas-vapour mixture to flow directly into the flow tube of the SIFT-MS instrument (Ref 27). The flow rate of the sample into the instrument is controlled by a heated calibrated capillary and is approximately 40 mL min⁻¹ so the typical headspace volume of 135 mL can be sampled for about 30 seconds before the pressure in the bottle is significantly reduced. This flow rate is sufficiently small that the pressure reduction in the sealed bottle is only small and insufficient to significantly distort the analyses. This phenomenon has been discussed in a previous publication (Ref 27). The trace compounds of the sample headspace are ionised by the appropriate precursor/reagent ion species, which are simultaneously injected into the helium-buffered reaction flow tube of the instrument. The precursor ions, which are always H3O⁺, NO⁺ or O2⁺, do not react significantly with the major components of air in the headspace sample, minimising interference from such compounds as nitrogen, oxygen and argon, and the resulting product ions are characteristic of the trace volatile analyte molecules. The product ions and the precursor ions are then analysed by a quadrupole mass spectrometer/detection system. Throughout the present study the instrument was operated in the multiple ion monitoring (MIM) mode, during which the desired precursor ion and its hydrates (Ref 33) and the characteristic product ions, are continuously monitored. This system, in combination with the previously compiled kinetics data for the reactions between the precursor ions and neutral molecules (Refs 34,35 rapidly allows the simultaneous absolute quantification of the concentrations of several selected volatile compounds of interest. The simultaneous detection and analysis of AA and DMS in a mixture presents a peculiar problem to SIFT-MS because of the overlap of characteristic product ions on which the analysis depends. However, the ion chemistry involved has been studied in detail and the kinetics database entries required for the accurate analyses of these two compounds by SIFT-MS have been constructed by following the guidelines given by previous studies (refs 34,35). In brief, AA was analysed using H3O⁺ precursor ions and the unique protonated AA characteristic product ion at m/z 45, as described in detail in a recent paper (Ref 36). Since protonated DMS forms a monohydrate at m/z 81, the dihydrate of protonated AA could not be used as an analytical ion for AA in the presence of DMS, as has been used previously (Ref 35). The unambiguous analysis of DMS in the presence of AA was achieved using NO precursor ions, again, as discussed in ref. 34 and 36.

### Viability Assay

In order to assess any changes in the viability of hepG2 cells due to the method of analysis, which requires the cells to be sealed inside a glass bottle for around 16 hours, cells were cultured to near-confluence using the untreated medium described earlier, and observed using a live-dead staining (Sigma, UK), and confocal microscopy. 1.5(10⁷) cells were suspended in 15 mL of DMEM medium containing 200 μM DEAB or 20 μM DSF, prepared as described previously, as well as an untreated sample with no inhibitors or DMSO present. The cell-suspensions were then sealed inside 150 mL glass bottles and incubated at 37° C. for 16 hours. The suspensions were then removed from the bottles and assessed by the same live-dead staining.

To test the effects of the ALDH inhibitors on cell viability, hepG2 cells and hMSCs were cultured to near-confluence in 96-well plates in their respective media, and treated with ALDH inhibitors for 16 hours. An ATPLite kit (Perkin-Elmer, UK) was used to quantify the ATP concentrations, according to the manufacturer's instructions. A Synergy 2 spectrophotometer (BioTek, UK) was employed to detect luminescence levels.

### EXAMPLE 2

### Analysis of the Background Culture Media Headspace

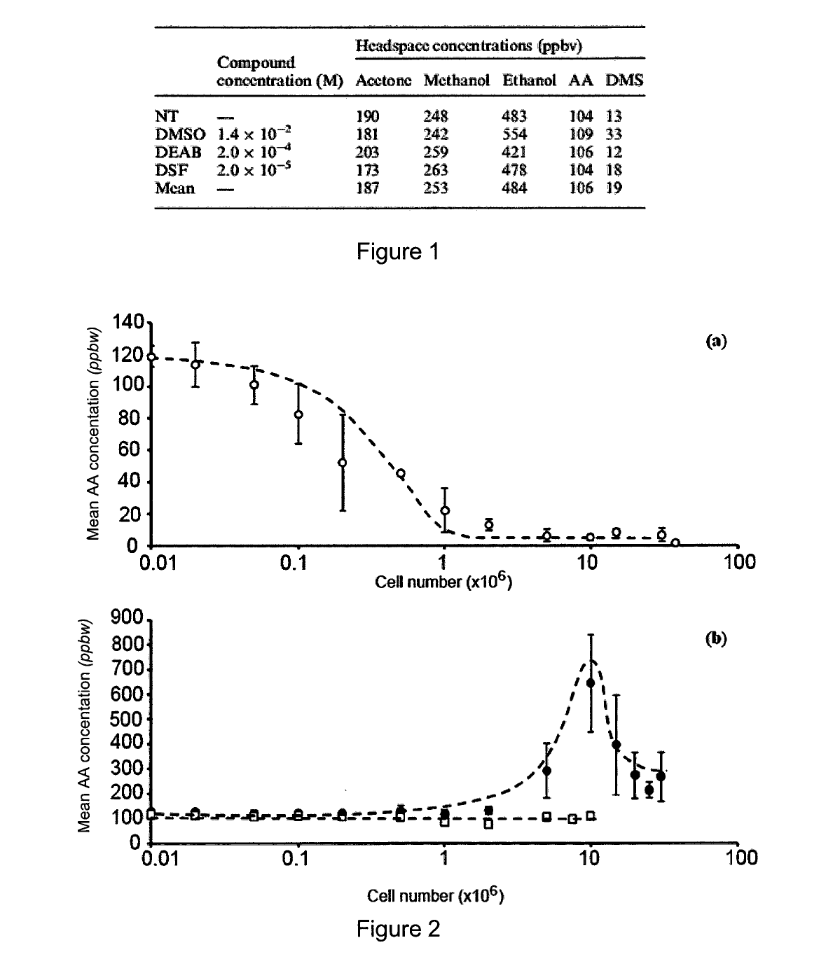

A first stage in these studies was to establish the background concentrations of the common metabolites in the headspace of the DMEM medium that was used exclusively in these cell culture studies. Thus, the concentrations of acetone and ethanol, which have previously been shown to largely originate from the foetal bovine serum (FBS) (Ref 22) as well as methanol, were routinely measured as reference controls, alongside acetaldehyde (AA) and DMS, which were the primary focus of this study. The potential influence of DMSO, DEAB and DSF additions to the DMEM medium was assessed. FIG. 1 indicates the headspace concentrations of the volatile compounds in DMEM media control samples with and without the addition of the varying concentrations of DMSO, DEAB and DSF. As can be seen, there is no discernible change in the headspace concentrations due to the addition of the three compounds beyond the spread in the measured values.

### EXAMPLE 3

### AA Measurements in the Headspace of HepG2 Cultures

Removal of AA from DMEM medium by cellular action. An initial probing experiment was conducted in which pure AA was added to 50 mL of the composite DMEM culture medium alone contained in three of our standard 150 mL bottles containing 20 million hepG2 cells prior to the 16-hour incubation period at 37° C. Identical medium samples without cells were also analysed (as described above) to obtain the starting (background) headspace AA concentration. The starting AA concentrations were measured as 59, 396 and 990 parts-per-billion by volume (ppbv), or 0.4, 2.8 and 7.2 μM the liquid phase, which were reduced by approximately 90% in the presence of the 20 million hepG2 cells, to 20, 48 and 118 ppbv respectively. The headspace concentrations of acetone, ethanol and methanol were unchanged by the presence of the added AA, the measured mean values, in ppbv, being 264 (274); ethanol 208 (212); and methanol 73 (67), the concentration without cells shown in parentheses.

The loss of AA from the DMEM medium was also investigated as the number of hepG2 cells in the medium was varied, beginning at the low AA level in 15 mL of medium that is partly due only to the presence of the FBS. The results of these studies are illustrated in FIG. 2(a) over the wide range of cell numbers from 1(10⁴) through 3(10⁷) and following 16 hours incubation at 37° C. The initial headspace concentration of AA of about 120 ppbv is rapidly reduced to almost background levels for about 1(10⁶) cells, which rapidly levels off with increasing cell numbers up to 3(10⁷) and asymptotically approaches zero ppbv. This trend can be qualitatively explained by following Michaelis-Menton enzyme kinetics (Ref 1) which has been used here to produce a model plot of the AA headspace concentration at the different cell numbers covered in this study. This model assumes that AA formation and loss is entirely due to the ethanol metabolism pathway: ethanol/AA/acetate, which is further assumed to proceed entirely by the actions of alcohol dehydrogenases -1B1, -1C1 , -1C2 and aldehyde dehydrogenases ALDH1 B1 and ALDH2. The implication of this metabolic pathway is that AA is constantly produced, and at the same time removed by the aforementioned enzymes. The Michaelis-Menton constants (K_{M}) and maximum enzyme reaction rates (Vₘₐₓ) values were obtained from the literature (Refs 3,4,37). Thus, assuming initial concentrations of 500 ppbv (44 μM, liquid phase) ethanol and 100 ppbv (0.7 μM, liquid phase) AA in the medium, and that each hepG2 cell contains 5000 combined alcohol dehydrogenase -1B1, -1C1, -1C2 molecules and 8000 combined ALDH1B1 and ALDH2 molecules, then 10⁶ cells are calculated to consume 80% (net) of the AA from the culture medium over 16 hours, but only 1% of the ethanol. In this way the variation in AA headspace concentration can be obtained for other cell numbers and the dashed plot in FIG. 2(a)is a representation of the predicted variation. This approach assumes that vapour phase/liquid phase equilibrium is established and that Henry's Law applies to this complex system (Henry's Law coefficients have been derived previously in SIFT-MS experiments—Ref 27). As can be seen, the model plot approximately follows the data points from which we conclude that enzymatic activity is responsible for the loss of AA from the medium. Finally, it should be appreciated that the Henry's Law coefficients for ethanol and AA are greatly different at 37° C., favouring the partition of AA into the headspace by a factor of about 12 times (Ref 27). Hence, the change in the ethanol headspace concentration for the observed changes in the AA headspace concentration will be relatively small and not discernible within the measurement uncertainty.

Inhibition of ALDH with DEAB

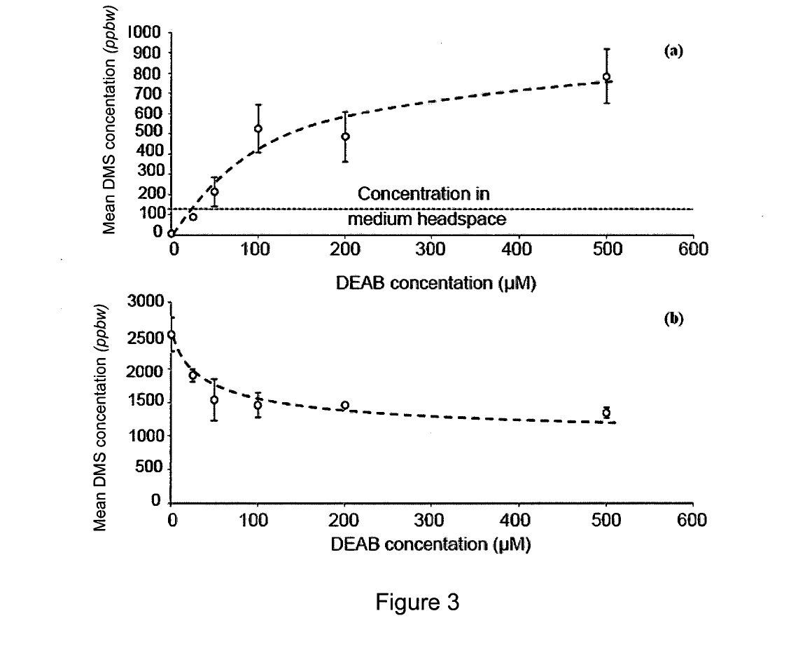

In order to investigate the loss of AA via enzymatic processes, experiments were carried out in which the ALDH inhibitor DEAB was added at different concentrations to the DMEM medium containing 1.5(10⁷) hepG2 cells and the combinations were incubated for 16 hours at 37° C. after which the headspace AA concentrations were measured using SIFT-MS. The results obtained are shown in FIG. 3(a), where a general increase in the AA concentration is seen with increasing DEAB in the cell culture media; the rate of increase apparently slowing down at the higher DEAB concentrations.

Headspace AA concentrations were also measured in the presence of a fixed concentration of DEAB as the number of cells in the medium was varied. For these studies a mid-range concentration of DEAB of 200 μM was chosen from a consideration of the data in FIG. 3(a) and the cell number was varied between 1(10⁴) and 5(10⁶). The results of these experiments are given in FIG. 2(b). The first point to notice is that there is not a reduction in the AA concentration even at the lowest cell number as is seen in the data in FIG. 2(a); in fact, there is a small increase compared to the DMEM medium alone. Then as the cell number approaches 1(10⁶) a clear increase in the headspace AA occurs and a peak of around 600 ppbv (4 μM in the liquid phase) is apparent at about 1(10⁷) cells with a subsequent reduction as the cell number is further increased. It is worthy of note that in these DEAB intervention experiments the acetone, methanol and ethanol present in the headspace of each cell culture did not change with cell number.

Inhibition of ALDH with Disulfiram

Similar experiments to those above were carried out to study the influence of the enzyme inhibitor disulfiram (DSF) on AA production/loss. The DSF was added to the cell cultures at a maximal concentration of 20 μM (see below in the Sample preparation section). Then the headspace AA concentrations were measured for hepG2 cell cultures over the cell number range from 1(10⁴) to 1(10⁷) and the results are shown in FIG. 2(b). It is clear that no increase in the AA levels occurs; rather a small, continuous decrease from the DMEM headspace levels is observed and not the small but certain increase seen for DEAB addition over the same cell number range. However, this is likely due to the much lower concentration of DSF that was possible compared to that for DEAB (see Sample preparation section).

### EXAMPLE 4

### Dimethyl Sulphide (DMS) Measurements in HepG2 Cultures

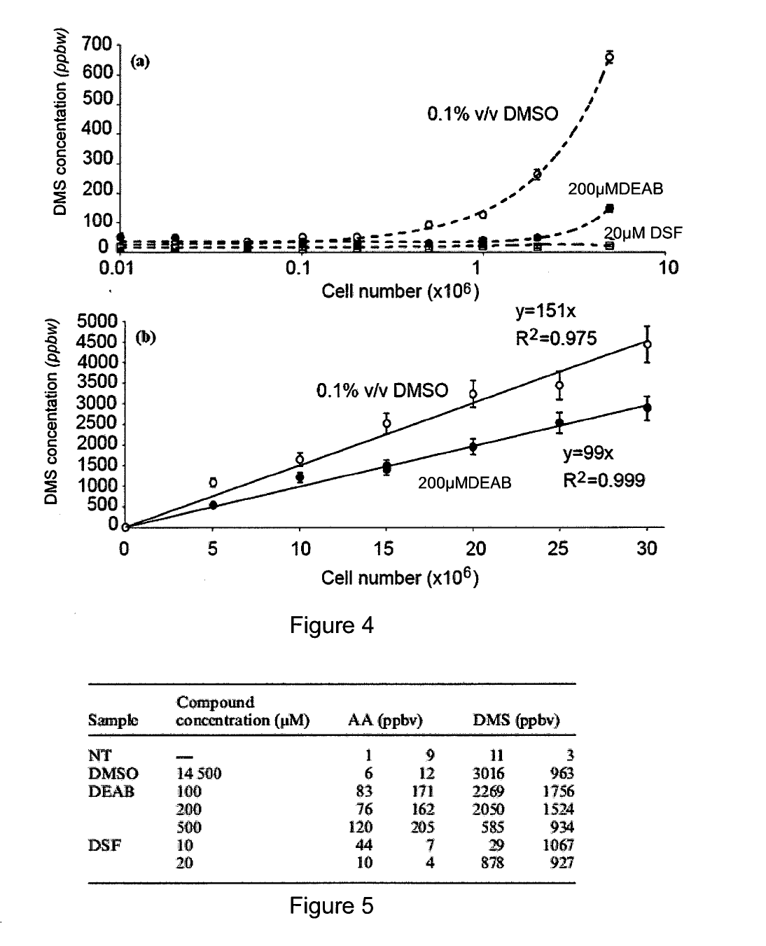

DMSO reduction to DMS by cellular action and inhibition by DEAB and DSF. It was observed during the experiments that, following overnight incubation, DMS was present in the headspaces of the cell-containing samples at parts-per-million by volume (ppmv) levels where 0.1% v/v DMSO was initially present in the medium as a solvent or control. This translates to liquid phase concentrations of >300 nM, by Henry's Law. Thus, the effects of ALDH inhibitors DEAB and DSF on the DMSO to DMS reduction reactions in hepG2 cells were simultaneously investigated with the AA measurements. As before, the first experiment involved varying the DEAB concentration (dissolved in 0.1% v/v DMSO) in cell cultures containing 1.5(10⁷) hepG2 cells. The results of these experiments are shown in FIG. 3(b), where it can be seen that from 0 to 25 μMDEAB, there is a 30% decline in headspace DMS, but little further reduction with increasing DEAB concentration. The influence of cell numbers on the DMS production from DMSO, and on the inhibitory effects of DEAB, were also investigated. As before, the DEAB concentration was 200 μM in all samples, with the cell numbers varied. The plot shown in FIG. 4(a) provides further evidence that DEAB inhibits DMS production. At cell numbers less than 5(10⁵), when only DMSO is present and the ALDH inhibitors are absent, DMS production is negligible (1 ppbv or less in the headspace), but between 5(10⁵) and 5(10⁶), the DMS concentration clearly increases. However, when 200 μM DEAB is also present, DMS production is clearly reduced and at cell numbers less than 5(10⁶) cells there is little production of DMS above the level appropriate to the zero DEAB situation.

For cell numbers greater than 5(10⁶) up to 3(10⁷), DMS production continues to increase for both the zero DEAB situation (labelled DMSO) and for the 200 μM DEAB situation, but inhibition still occurs by the DEAB, as can be seen in FIG. 4(b). The DMS concentration appears to increase with cell number in a linear fashion, and the slope of the line declines by about 30% in the presence of the DEAB. A similar level of reduction is observed in the headspace concentration of DMS when DEAB is present, as shown in FIG. 3(b). These experiments were conducted from the same “stock” of cells, on the same day (i.e. not independent experiments).

When 20 μM DSF is added, no DMS production is observed, as can also be seen in FIG. 4(a). Thus, both DEAB and DSF are able to inhibit the reduction of DMSO to DMS by hepG2 cells.

### EXAMPLE 5

### Headspace Analyses of Human Mesenchymal Stem Cell Cultures

As a comparison with the hepG2 cell line, similar experiments (see section: Sample preparation) were conducted using primary human bone marrow-derived mesenchymal stem cells (hMSCs). Thus, cultures of approximately 5(10⁶) hMSCs were in this case pre-treated for 24 hours during routine tissue flask culture with DEAB and DSF at the liquid concentrations given in FIG. 5. The results of these exploratory experiments, also given in FIG. 5, suggest that only DEAB is an effective inhibitor of ALDH in the hMSCs, under these conditions, and it is also effective at preventing DMS production from DMSO, particularly at the higher DEAB concentration of 500 μM. DSF, on the other hand, apparently has little or no effect on the ability of the hMSCs to metabolise AA, but it has an inhibiting effect on DMS production from DMSO, although the lower concentration of DSF, relative to the DEAB experiments, may again be a contributing factor to the observed metabolism of AA.

### EXAMPLE 6

### Assessment of Viability in HepG2 and hMSC Cultures

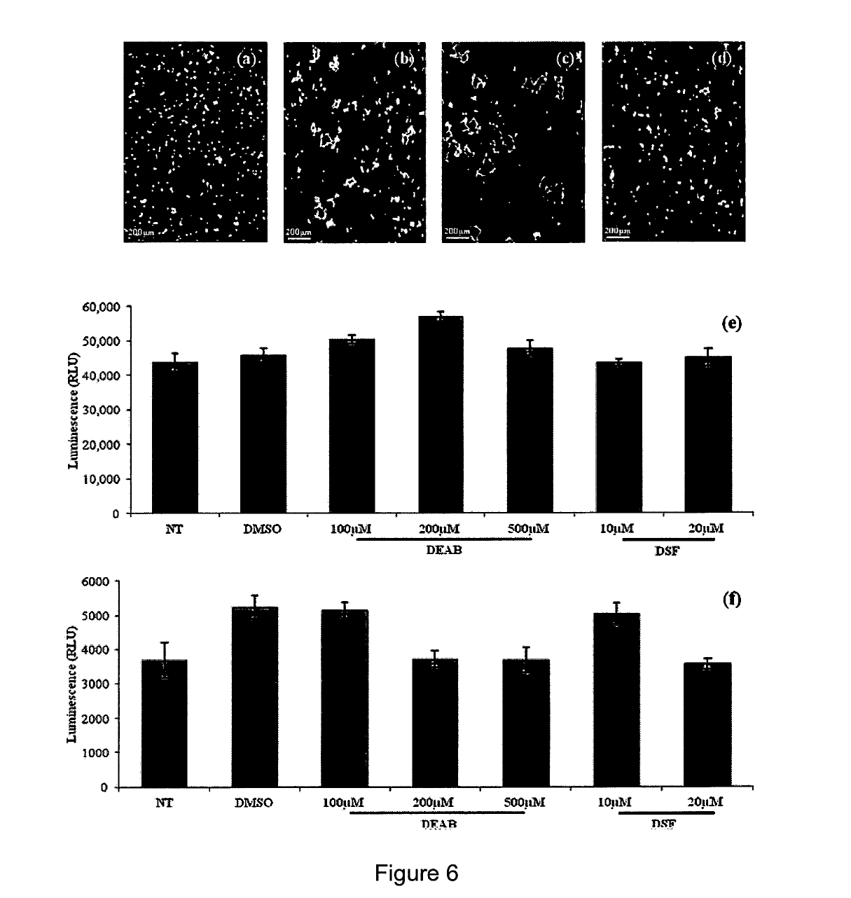

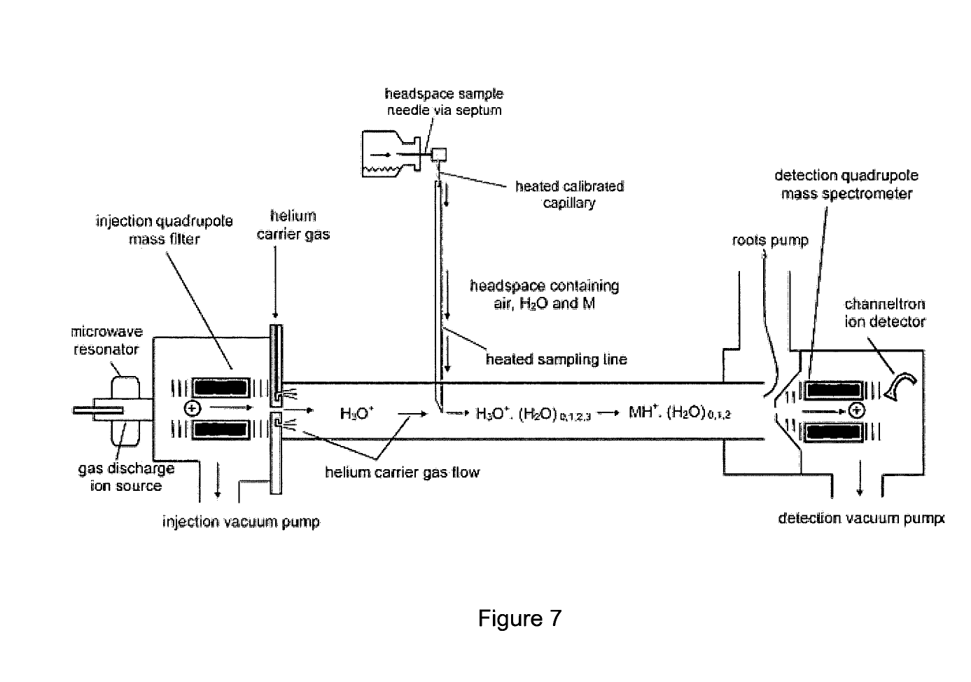

The potential effects of the overnight incubation in the sealed bottle on the viability of the hepG2 were observed using live-dead staining, the resulting images being shown in FIG. 6(a)-(d). Here it is clear that the method of keeping the cells sealed for a period of 16 hours, with and without the presence of DEAB or DSF, caused little or no decline in the viability of the hepG2 cells cultured. The effects of treating the cells with the ALDH inhibitors on the viabilities of hepG2 and hMSC cells were also assessed and the resulting luminescence measurements are shown in FIG. 6 (e) and (f). These indicate that that the inhibitors had no clear effect on the viability of either cell type. However, we have not thoroughly investigated the effects of this protocol of analysis on the condition of the cells, and it is surely true that these adherent cell types will initially proliferate at a decreased rate on transfer to suspension culture conditions and will tend to form aggregates. These could affect cell behaviour. The essential point is that the cells were all exposed to ostensibly identical conditions, and the inhibitory effects of DEAB and DSF were clear.

### EXAMPLE 7

### MG63 Cells Attached to Glass Substrate

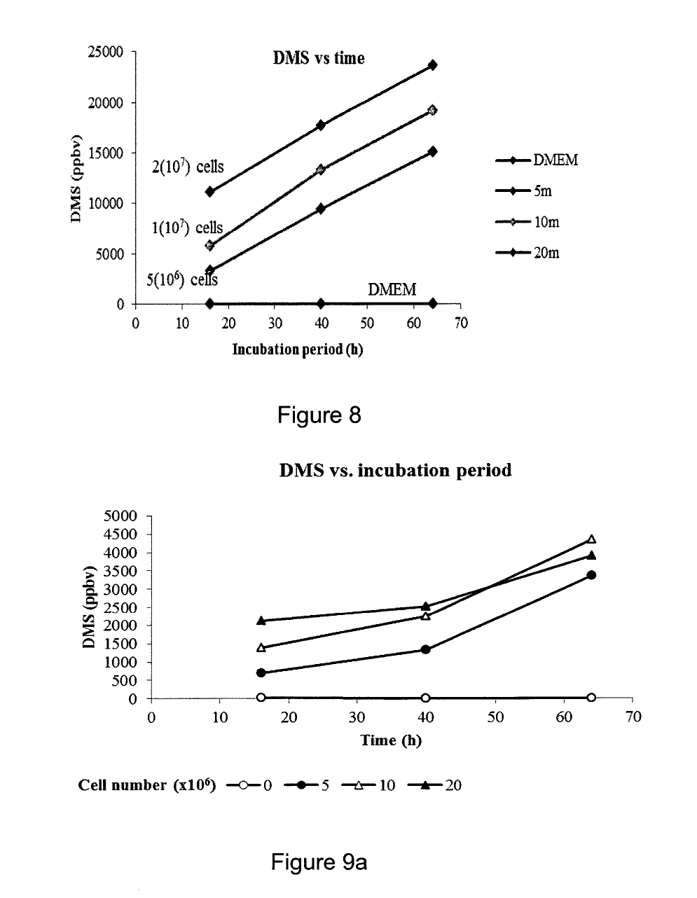

In this experiment, 11 μL of DMSO was added to 11 mL of DMEM with 5, 10 and 20 million MG-63 cells attached to glass substrate prior to the initial 16-hour incubation period with headspace analyses performed after 16, 40 and 64 hours. Results are shown in FIG. 8.

### EXAMPLE 8

### MG63 Cells in Collagen

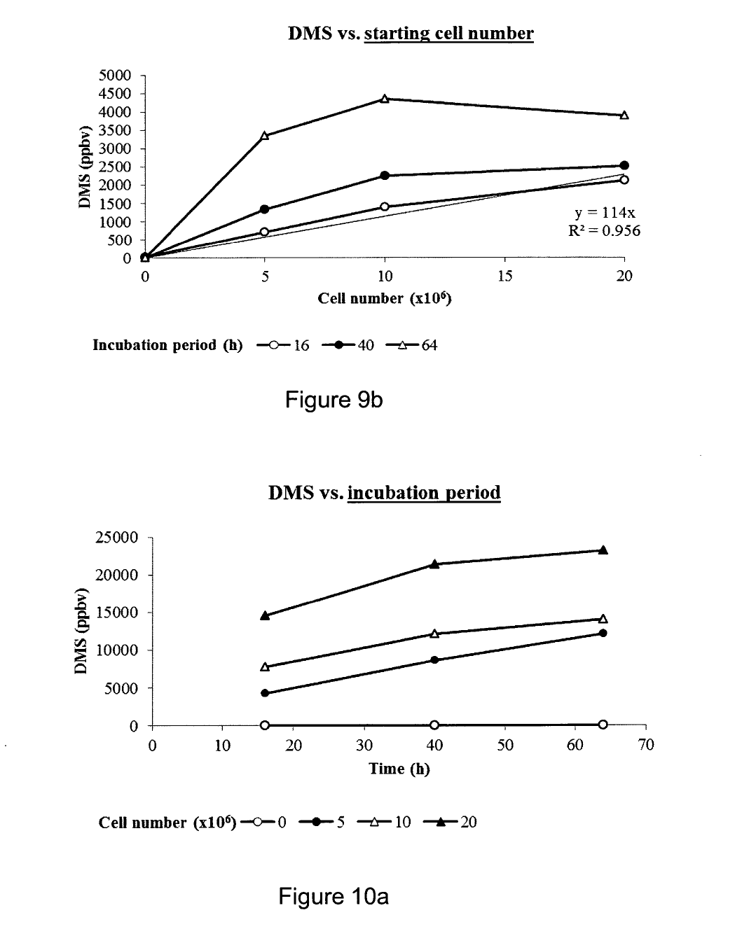

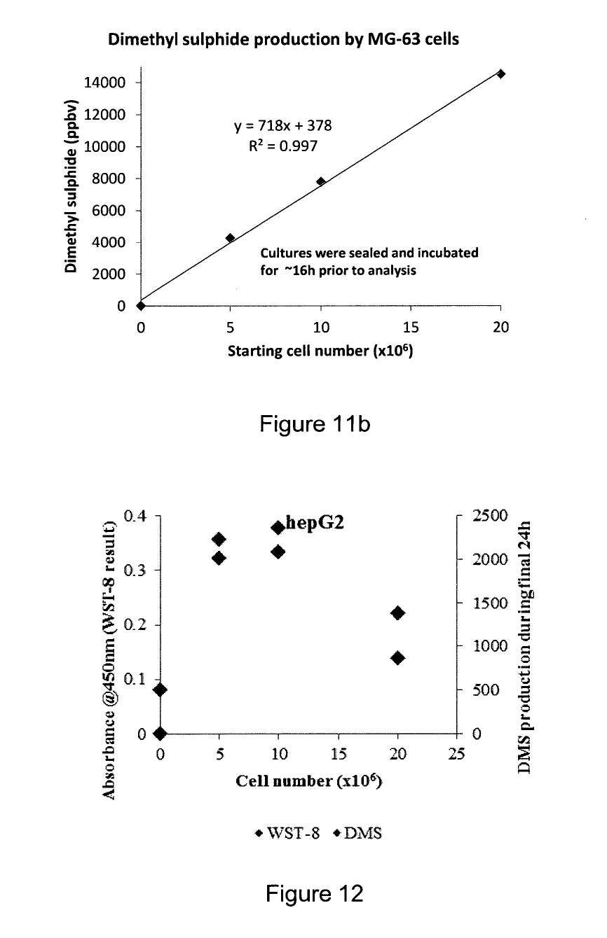

In these experiments, MG63 cells were contained in 5.5 mL of collagen, to which 5.5 mL of DMEM medium was added. Both the medium and collagen scaffold contained 0.1% v/v DMSO. The headspaces of cultures containing MG-63 osteosarcoma cells and hepG2 hepatocellular carcinoma cells were analysed following 16, 40 and 64 hours. Results are shown in FIGS. 9 and 11.

### EXAMPLE 9

### HepG2 Cells in Collagen

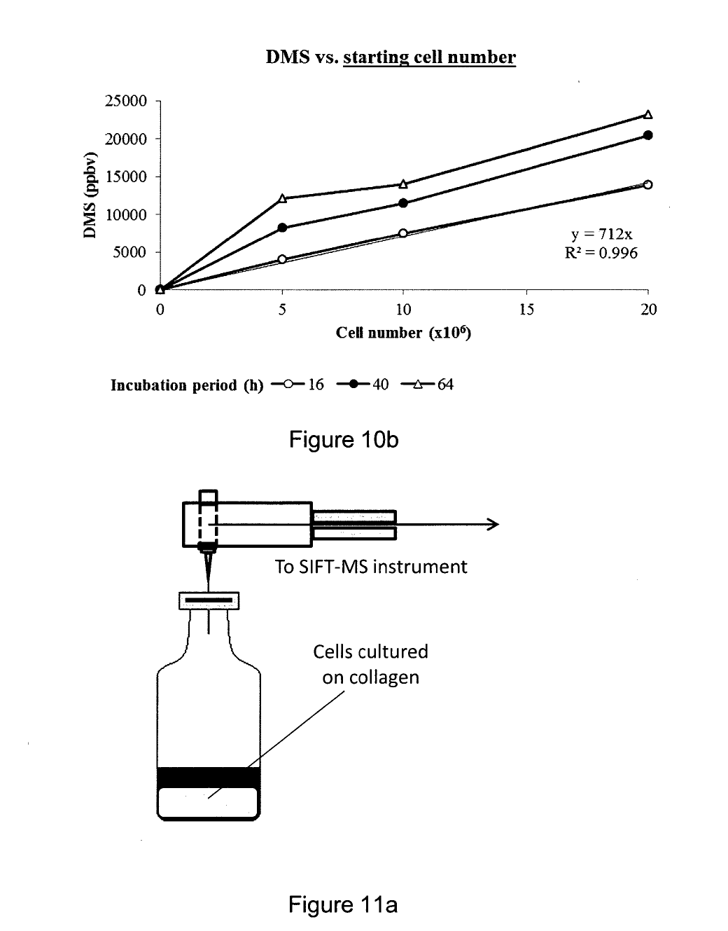

In these experiments, HepG2 cells were contained in 5.5 mL of collagen, to which 5.5 mL of DMEM medium was added. Both the medium and collagen scaffold contained 0.1% v/v DMSO. The headspaces of cultures containing MG-63 osteosarcoma cells and hepG2 hepatocellular carcinoma cells were analysed following 16, 40 and 64 hours. Results are shown in FIG. 10.

### EXAMPLE 10

### Comparison of DMS Production and WST-8 (Marker of Cell Proliferation)

WST-8 is used as a measure of cell proliferation/activity or cell number. The WST-8 compound ([2-(2-methoxy-4-nitrophenyI)-3-(4-nitrophenyl)-5-(2,4-disulfophenyl)-2H-tetrazolium, monosodium salt]) is added to the bottle containing the cells (in collagen) and medium and incubated for 4 hours. As with the MTT assay, the cells convert the compound to a dye (in this case a formazan) by dehydrogenase enzymes in the cells. Following this 4-hour incubation, 100 μL of the supernatant is transferred to a 96-well plate, and the absorbance is measured at 450nm. The results are shown in FIG. 12 in comparison to the levels of DMS production measured using SIFT MS during the final 24 h of culture (DMS₆₄ₕ-DMS₄₀ₕ).

### EXAMPLE 11

### Discussion

This study has described how SIFT-MS can be used to non-invasively identify and accurately quantify volatile metabolite compounds present in the headspace above immortalised cell lines and primary derived cell cultures and thereby analyse metabolic processes occurring in vitro. Crucially, it was observed that the hepG2 cells, specifically the functional ALDH present within the cells, metabolised the AA, effectively removing it from the medium. However, when the ALDH enzyme inhibitor DEAB was present in the cell cultures the headspace AA increased, seemingly because the DEAB concentration was sufficient to inhibit the action of the cellular ALDH in removing AA, according to the general equation below.

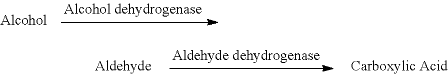

On the other hand, alcohol dehydrogenase was not inhibited in producing AA, and consequently the liquid phase/vapour phase AA increases. This explains why DEAB appeared to cause the accumulation of AA, as opposed to simply preventing the loss of the compound, as was the case for DSF. The spread in the AA levels measured in each experiment are presumably due to batch-to-batch variations in the cell status (cell cycle), which are likely linked to enzyme expression levels (Ref 38). That the AA level does not fall at the lower cell numbers (see FIG. 2(a)) is the result of the inhibiting effect of the DEAB molecules. At the highest cell densities the ALDH molecular density could become excessive leaving the DEAB insufficient to prevent the loss of AA, which is seen to decrease towards the initial DMEM value. In short, the accumulation and loss of AA in this way is the key to understanding the form of the data curve shown in FIG. 2(b). It is worth mentioning that acetic acid can be quantified by SIFT-MS (Ref 39) but because the pH of the cell culture media (typically 7.5) any acetic acid formed exists largely as non-volatile acetate ions. However, several biogenic molecular species, including methanol, ethanol, acetone and AA were observed in the headspace of the DMEM medium used exclusively for these studies. Acetone and ethanol were previously reported to originate largely in the FBS, which is routinely added to the cultures (Ref 22). The presence of these compounds was confirmed in these latest studies.

The reduction of DMSO to DMS by the hepG2 cells was observed, which was apparently also inhibited by the presence of DEAB and DSF in the hepG2 cell cultures, which, to the authors' knowledge has not been reported previously. It has previously been reported that DMSO can be reduced by the actions of the enzyme methionine sulfoxide reductase A (MsrA)(Refs 40-42). The presence of this enzyme in the hepG2 cells and hMSCs used in this study has been confirmed by quantitative real-time polymerase chain reaction (PCR) (data not shown). It is possible that DEAB and DSF directly inhibited the actions of the cellular MsrA, for example by interacting with the cysteine residues of the enzyme active site (Ref 43). Alternatively, the inhibitors could have indirectly affected the activity of this enzyme by causing aldehyde concentrations to increase in the media, although no linear relation is evident from the plots in FIGS. 2(b) and 4. MsrA is believed to protect tissue from oxidative damage, and may be related to the ageing process. Diminished MsrA activities have also been reported in the brains of Alzheimer's disease patients (Ref 44) and there is evidence to suggest that the enzyme protects dopaminergic cells from Parkinson's disease related damage (Ref 45). To the authors' knowledge, the possibility of a link between ALDH/AA and MsrA function/activity levels has not been reported in the literature. This may have implications for the use of DSF (Antabuse) for the treatment of alcohol and cocaine addictions, mentioned previously (Ref 11). Also, given that ALDH has also been linked with the development of both Parkinson's (Ref 5) and Alzheimer's (Ref 10) diseases, these findings may be significant in the pathology of these diseases.

In summary, we have demonstrated that SIFT-MS can be used for the measurement of volatile compounds emitted by cell cultures, and that these measurements can be used to study cellular activity, including that of specific intracellular enzymes, and even enzyme kinetics. The scope of this technique is not limited to the study of AA metabolism by ALDH, as is demonstrated by the finding that the cell-types studied both reduced DMSO to DMS. Provided that the substrates and/or products of enzymatic reactions are volatile, the described techniques could certainly be applied for the analysis of the metabolic activity of other cell types, including microbial cells and animal cells, which can also be used to study time variations in volatile compound emissions and hence to follow the course of cellular activity and responses to chemical stimuli. Also, as is mentioned above, toxic AA and ALDH deficiencies have been linked to numerous diseases, such as ethanol-induced cancers (Ref 6) alcoholic liver disease (Ref 7) and ischaemic tissue diseases (Ref 9). The simple, rapid and non-invasive analytical methods described in this study could surely find utility in drug screening, which could aid in the treatment or prevention of these and other diseases.

### REFERENCES

1. J. T. Berg and L. Stryer, Biochemistry, W.H. Freeman and Company, New York, USA, 2002.
2. D. Conklin, R. Prough and A. Bhatanagar, Mol. BioSyst., 2007, 3, 136-150.
3. M. F. Wang, C. L. Han and S. J. Yin, Chem.-Biol. Interact., 2009, 178, 36-39.
4. D. Stagos, Y. Chen, C. Brocker, E. Donald, B. C. Jackson, D. J. Orlicky, D. C. Thompson and V. Vasiliou, Drug Metab. Dispos., 2010, 38, 1679-1687.
5. D. Gaiter, S. Buervenich, A. Carmine, M. Anvret and L. Olson, Neurobiol. Dis., 2003, 14, 637-647.
6. J. S. Moreb, Curr. Stem Cell Res. Ther., 2008, 3, 237-246.
7. C. J. Eriksson, Alcohol.: Clin. Exp. Res., 2001, 25, 15S-32S.
8. T. Mello, E. Ceni, C. Surrenti and A. Galli, Mol. Aspects Med., 2008, 29, 17-21.
9. H. S. White, L. Smith, T. Gentry and A. E. Balber, J. Stem Cell Res. Ther., 2011, 51, DOI: 10.4172/2157-7633.S1-001.
10. S. Ohta, I. Ohsawa, K. Kamino, F. Ando and H. Shimokata, Ann. N. Y. Acad. Sci., 2004, 1011, 36-44.
11. K. M. Carroll, L. R. Fenton, S. A. Ball, C. Nich, T. L. Frankforter, J. Shi and B. J. Rounsaville, Arch. Gen. Psychiatry, 2004, 61, 264-272.
12. P. Marcato, C. A. Dean, D. Pan, R. Araslanova, M. Gillis, M. Joshi, L. Helyer, L. Pan, A. Leidal, S. Gujar, C. A. Giacomantonio and P. W. K. Lee, Stem Cells, 2011, 29, 32-45.
13. C. Yu, Z. Yao, J. Dai, H. Zhang, J. Escara-Wilke, X. Zhang and E. T. Keller, In Vivo, 2011, 25, 69-76.
14. M. P. Kim, J. B. Fleming, H. Wang, J. L. Abbruzzese, W. Choi, S. Kopetz, D. J. McConkey, D. B. Evans and G. E. Gallick, PLoS One, 2011, 6, e20636.
15. J. S. Moreb, D. Mohuczy, B. Ostmark and J. R. Zucali, Cancer Chemother. Pharmacol., 2007, 59, 127-136.
16. J. S. Moreb, D. Ucar, S. Han, J. K. Amory, A. S. Goldstein, B. Ostmark and L. J. Chang, Chem.-Biol. Interact., 2012, 195, 52-60.
17. D. Gen, D. Brayton, B. Shahandeh, F. L. Meyskens and P. J. Farmer, J. Med. Chem., 2004, 47, 6914-6920.
18. S. S. Brar, C. Grigg, K. S. Wilson, W. D. J. Holder, D. Dreau, C. Austin, M. Foster, A. J. Ghio, A. R. Whorton, G. W. Stowell, L. B. Whittall, R. R. Whittle, D. P. White and T. P. Kennedy, Mol. Cancer Ther., 2004, 3, 1049-1060.
19. M. V. Lioznov, P. Freiberger, N. Kroger, A. R. Zander and B. Fehse, Bone Marrow Transplant., 2005, 35, 909-914.
20. J. S. Moreb, J. R. Zucali, B. Ostmark and N. A. Benson, Cytometry, Part B, 2007, 72B, 281-289.
21. D. Smith, T. Wang, J. Sule-Suso, P. Spanel and A. J. El Haj, Rapid Commun. Mass Spectrom., 2003, 17, 845-850.
22. J. Sule-Suso, A. Pysanenko, P. Spanel and D. Smith, Analyst, 2009, 134, 2419-2425.
23. H. W. Shin, B. Umber, S. Meinardi, S. Y. Leu, F. Zaldivar, D. Blake and D. Cooper, J. Transl. Med., 2009, 7, 31.
24. C. Brunner, W. Szymczak, V. Hollriegl, S. Mortl, H. Oelmez, A. Bergner, R. M. Huber, C. Hoeschen and U. Oeh, Anal. Bioanal. Chem., 2010, 397, 2315-2324.
25. W. Filipiak, A. Sponring, T. Mikoviny, C. Ager, J. Schubert, W. Miekisch, A. Amann and J. Troppmair, Cancer Cell Int., 2008, 8, 17.
26. A. Sponring, W. Filipiak, T. Mikoviny, C. Ager, J. Schubert, W. Miekisch, A. Amann and J. Troppmair, Anticancer Res., 2009, 29, 419-426.
27. P. Spanel, A. M. Diskin, S. M. Abbott, T. Wang and D. Smith, Rapid Commun. Mass Spectrom., 2002, 16, 2148-2153.
28. K. Dryahina, F. Pehal, D. Smith and P. Spanel, Int. J. Mass Spectrom., 2009, 286, 1-6.
29. D. Smith and P. Spanel, Mass Spectrom. Rev., 2005, 24, 661-700.
30. P. Spanel, K. Dryahina and D. Smith, Int. J. Mass Spectrom., 2006, 249-250, 230-239.
31. T. W. E. Chippendale, P. Spanel and D. Smith, Rapid Commun. Mass Spectrom., 2011, 25, 2163-2172.
32. D. Smith and P. Spanel, Analyst, 2011, 136, 2009-2032.
33. P. Spanel and D. Smith, J. Phys. Chem., 1995, 99, 15551-15556.
34. P. Spanel and D. Smith, Int. J. Mass Spectrom., 1998, 176, 167-176.
35. P. Spanel and D. Smith, J. Breath Res., 2008, 2, 046003.
36. D. Smith, T. W. E. Chippendale and P. Spanel, Curr. Anal.Chem., in press.
37. S. L. Lee, H. T. Shih, Y. C. Chi, Y. P. Li and S. J. Yin, Chem.-Biol. Interact., 2011, 191, 26-31.
38. M. D. C. Cordoba-Pedregosa, J. M. Villalba, D. Gonzalez-Aragon, R. I. Bello and F. J. Alcain, Anticancer Res., 2006, 26, 3535-3540.
39. A. Pysanenko, P. Spanel and D. Smith, Int. J. Mass Spectrom., 2009, 285, 42-48.
40. J. Moskovitz, H. Weissbach and N. Brot, Proc. Natl. Acad. Sci. U.S.A., 1996, 93, 2095-2099.
41. H. Weissbach, L. Resnick and N. Brot, Biochim. Biophys. Acta Protein Proteomics, 2005, 1703, 203-212.
42. G. H. Kwak, S. H. Choi, J. R. Kim and H. Y. Kim, BMB Rep., 2009, 42, 580-585.
43. W. T. Lowther, N. Brot, H. Weissbach, J. F. Honek and B. W. Matthews, Proc. Natl. Acad. Sci. U.S.A., 2000, 97, 6463-6468.
44. S. P. Gabbita, M. Y. Aksenov, M. A. Lovell and W. R. Markesbery, J. Neurochem., 1999, 73, 1660-1666.
45. F. Liu, J. Hindupur, J. L. Nguyen, K. J. Ruf, J. Zhu, J. L. Schieler, C. C. Bonham, K. V. Wood, V. J. Davisson and J. C. Rochet, Free Radical Biol. Med., 2008, 45, 242-255.

1. A method for determining the number of cells in an in vitro culture of cells, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, measuring the DMS produced by the cultured cells.

2. A method of determining a change in the number of cells contained in a cell culture, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, measuring the DMS produced by the cultured cells at a first time point and determining the number of cells in the culture at said first time point, measuring the DMS produced by the cultured cells at a second time point and determining the number of cells in the culture at said second time point.

3. A method of determining the ability of a test compound or test condition to cause cell death of cells cultured in vitro, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, contacting the cells with a test compound or subjecting the cells to a test condition, measuring the DMS produced by the cultured cells.

4. A method of determining the ability of a test compound or test condition to enhance proliferation of cells cultured in vitro, the method comprising culturing cells in the presence of dimethyl sulphoxide (DMSO), or in the presence of a substrate capable of enzymatic conversion to dimethyl sulphide (DMS) by an enzyme or enzymes present in the cells, for a period of time sufficient for the cells to produce DMS, contacting the cells with a test compound or subjecting the cells to a test condition, measuring the DMS produced by the cultured cells.

5. The method of any one of claims 2 to 4, wherein the cells are contacted with a test compound or are subjected to a test condition after having been cultured for a period of time sufficient for the cells to produce DMS and the DMS produced by the cells is measured before and after contact of the cells with the test compound or before and after being subjected to the test condition to determine a change in the level of DMS produced by the cultured cells.

6. The method of any one of claims 2 to 5, wherein the cells are contacted with a test compound or are subjected to a test condition during culture of the cells for a period of time sufficient for the cells to produce DMS.

7. A method for determining the number of cells in an in vitro culture of cells, the method comprising measuring the dimethyl sulphide (DMS) produced by the cultured cells.

8. An in vitro method of monitoring or measuring cell proliferation comprising detecting dimethyl sulphide (DMS) produced by cells in vitro.

9. An in vitro method of monitoring or measuring cell death comprising detecting dimethyl sulphide (DMS) produced by cells in vitro.

10. The method of any one of claims 1 to 9, wherein the cells are mammalian or human cells.

11. The method of any one of claims 1 to 10, wherein the cells are capable of reducing DMSO to DMS.

12. The method of any one of claims 1 to 10, wherein the cells are capable of producing DMS when cultured in the presence of a substrate capable of enzymatic conversion to DMS by an enzyme or enzymes present in the cells.

13. The method of any one of claims 1 to 12, wherein measurement of DMS is conducted using Mass Spectrometry.

14. The method of claim 13, wherein the Mass Spectrometry technique used is SIFT-MS.

15. A kit comprising DMSO in a container and information indicating a plurality of DMS concentrations produced by a respective plurality of discrete numbers of cells per amount of DMSO added to the culture and per the time period of the culture with DMSO.

16. The kit of claim 15, wherein said information is provided on a computer readable medium.
